(19) United States
(12) Patent Application Publication
(10) Pub. No.: US 2023/0211911 A1
(43) Pub. Date: Jul. 6, 2023

(54) VACUUM LIQUID-FILLING NEEDLE AND PRESSURE RELIEF VALVE THEREFOR

(71) Applicant: SUZHOU REVEDA MEDICAL BIOTECH CO., LTD., Suzhou (CN)

(72) Inventors: Long LIU, Suzhou (CN); Ping YAN, Suzhou (CN); Yuan HUANG, Suzhou (CN); Qiuyu QU, Suzhou (CN)

(21) Appl. No.: 17/924,060

(22) PCT Filed: Jan. 26, 2021
(86) PCT No.: PCT/CN2021/073685

§ 371 Date: Nov. 8, 2022

(30) Foreign Application Priority Data
Jun. 16, 2020 (CN) 202010549503.X

(51) Int. Cl. B65B 39/00; B67C 3/26; A61M 5/168

(52) U.S. Cl. CPC B65B 39/004; B67C 3/26; A61M 5/16804

(57) ABSTRACT

A vacuum liquid-filling needle includes a needle shaft (2) and a pressure relief valve (3). The pressure relief valve is disposed within the needle shaft at a first end (21) thereof, and the needle shaft is able to connect at a second end (22) thereof to a pipe for supplying a solution to be filled. The pressure relief valve defines an internal cavity, in which a first slide valve and a second slide valve are disposed so as to be axially spaced apart from each other and both movable relative to the internal cavity. The valve body defines an inflow channel (33), an inflow port (34), an outflow port (35), an outflow channel (37) and exit apertures (38). Each of the inflow channel and the outflow channel is formed by a blind bore, and both the inflow channel and the outflow channel axially extend along the valve body. This vacuum liquid-filling needle can prevent undesired accidental efflux of the solution while providing depressurization and pressure stabilization effects during filling of the solution. Moreover, the solution can be sucked back in a quantitative manner, thus avoiding it from stringing at a dispensing port of the vacuum liquid-filling needle. This makes the filling process more stable, more controllable and suitable for use in precise filling in environments at high vacuum levels. Also disclosed is a pressure relief valve.

### TECHNICAL FIELD

The present invention relates to the technical field of liquid filling apparatuses and, in particular, to a vacuum liquid-filling needle and a pressure relief valve thereof.

### BACKGROUND

A liquid filling apparatus incorporates a liquid-filling needle as an important component mainly for quantitative extraction and injection of a liquid being filled, as widely seen in the pharmaceutical, chemical, food and beverage production industries.

A typical needle of this type is commonly used under an atmospheric pressure condition, or sometimes in a vacuum environment often with a low vacuum level. This is mainly because when the needle is placed in a high vacuum environment, a strong negative pressure therein tends to urge a liquid in a pipe in fluid communication with the needle to flow out of a tip of the needle, leading to contamination of the apparatus, or even degraded quality of filling. Even if the needle is provided with a certain sealing means, the liquid being filled would have to alternately expand and contract due to repeated switches between atmospheric and vacuum pressures. Consequently, the liquid tends to string at the needle tip, or even drip therefrom, affecting quality and stability of the filling process. Further, in addition to the negative pressure in the high vacuum environment, the liquid may also be driven by hydraulic power from a pump. As a consequence, it may exit the needle at an excessive speed, which tends to lead to splashes of the liquid being filled.

Chinese Patent Publication No. CN2546343Y mentions an anti-drip arrangement, which can be used with a liquid-filling needle to provide an anti-drip effect to the liquid being filled. It operates mainly by expansion and contraction of a rubber hose included therein, which provides some depressurization and “suck-back” effects on the liquid being filled. However, such depressurization and suck-back effects are provided in a relatively stochastic manner and considerably depend on variation of the rubber hose's elasticity over repeated use.

### SUMMARY OF THE INVENTION

The present invention seeks to provide a vacuum liquid-filling needle and a pressure relief valve thereof, which can prevent efflux or dripping of the liquid being filled, provide depressurization, pressure stabilization, pressure stabilization and quantitative suck-back effects, and enable filling of a solution in vacuum environments.

To this end, the present invention provides a pressure relief valve comprising a valve body defining an internal cavity, in which a first slide valve and a second slide valve are disposed so as to be axially spaced apart from each other and both movable relative to the internal cavity. The valve body defines an inflow channel, an inflow port, an outflow port and an outflow channel. Each of the inflow channel and the outflow channel is formed by a blind bore, and both the inflow channel and the outflow channel axially extend along the valve body. The inflow channel is brought into communication with the internal cavity through the inflow port, and the outflow channel is brought into communication with the internal cavity through the outflow port. The inflow port and the outflow port divide the internal cavity axially into a first cavity, an inter-cavity passage and a second cavity. The first slide valve is configured to be maintained in an initial configuration by a first elastic force at a first position where the first slide valve resides on the inflow port to block communication of the inter-cavity passage with the inflow channel and to, when subjected to a first axial pressure from a solution to be filled, which is greater than the first elastic force, move axially against the first elastic force from the first position toward the first cavity to bring the inter-cavity passage into communication with the inflow channel. The second slide valve is configured to be maintained in the initial configuration by a second elastic force at a second position where the second slide valve blocks communication of the inter-cavity passage between the first and second slide valves with the outflow channel and to, when subjected to a second axial pressure from the solution to be filled, which is greater than the second elastic force, move axially within the internal cavity against the second elastic force from the second position toward the second cavity to bring the inter-cavity passage between the first and second slide valves into communication with the outflow channel.

Preferably, the first slide valve may be further configured to, when the first axial pressure from the solution to be filled is below the first elastic force or disappears, return to the first position under an action of the first elastic force, and the second slide valve may be further configured to, when the second axial pressure from the solution to be filled is below the second elastic force or disappears, return to the second position under an action of the second elastic force.

Preferably, a damping aperture may be provided in a wall of the first cavity so as to bring the outflow channel into communication with the first cavity.

Preferably, the pressure relief valve may further comprise a first securing member and a second securing member, with the internal cavity having a third end and a fourth end, the first securing member fixed at the third end of the internal cavity, the second securing member fixed at the fourth end of the internal cavity, wherein: a first elastic member for providing the first elastic force is provided in the first cavity so as to abut against the first slide valve at one end and against the first securing member at a further end; and a second elastic member for providing the second elastic force is provided in the second cavity so as to abut against the second slide valve at one end and against the second securing member at a further end.

Preferably, the first slide valve may comprise a first slide valve body comprising a hollow first enclosing post extending axially within the first cavity, the first elastic member accommodated in the first enclosing post, and the second slide valve may comprise a second slide valve body comprising a hollow second enclosing post extending axially within the second cavity, the second elastic member accommodated in the second enclosing post.

Preferably, the first securing member may define a first groove for receiving the first enclosing post, and the second securing member may define a second groove for receiving the second enclosing post.

Preferably, a first protrusion may extend from the first groove toward the first slide valve, and the further end of the first elastic member is sleeved over the first protrusion, and a second protrusion may extend from the second groove toward the second slide valve, the further end of the second elastic member is sleeved over the second protrusion.

Preferably, a damping aperture may be provided in a wall of the first cavity so as to bring the outflow channel into communication with the first cavity, the damping aperture spaced from the first position by a distance that is greater than a distance from an open end of the first enclosing post to a bottom of the first groove.

Preferably, the valve body may be provided thereon with a first valve seat and a second valve seat, the first valve seat configured to maintain the first slide valve at the first position and block the first slide valve from approaching the second slide valve, the second valve seat configured to maintain the second slide valve at the second position and block the second slide valve from approaching the first slide valve.

Preferably, the valve body may be further provided therein with a suction aperture, through which the outflow channel comes into communication with the internal cavity, and the suction aperture is located between the outflow port and an outlet of the outflow channel.

Preferably, the first slide valve may be a piston or a diaphragm, and the second slide valve may also be a piston or a diaphragm.

Preferably, the first slide valve may be a piston with a bevel for enabling the solution to be filled to exert the first axial pressure on the piston.

Preferably, the pressure relief valve may further comprise exit apertures and an annular groove, wherein a number of the exit apertures is greater than that of the outflow channels, and the exit apertures communicate with the outflow channels through the annular groove.

The present invention also provides a needle for vacuum filling of a liquid comprising a needle shaft and a pressure relief valve. The pressure relief valve is disposed within the needle shaft at a first end thereof, and the needle shaft is able to connect at a second end thereof to a pipe for supplying a solution to be filled. The pressure relief valve is as defined above. The inflow channel is open at a location closer to the second end of the needle shaft, and the outflow channel is open at a location closer to the first end of the needle shaft.

Preferably, the pressure relief valve may be connected to the needle shaft by an interference fit or threadedly.

Preferably, a dispensing head may be provided at the first end of the needle shaft.

Preferably, the dispensing head may comprise a dispensing port having a cross-section in the shape of a square, a rectangle or circle, the square having a side length in the range of 10 mm to 200 mm, the rectangle having a length in the range of 10 mm to 400 mm and a width in the range of 0.05 mm to 200 mm, the circle having a diameter in the range of 10 mm to 200 mm.

Preferably, the dispensing head may be detachably connected to or integral with the needle shaft.

Preferably, the needle shaft may be provided at the second end thereof with an adapter for connecting to the pipe for supplying the solution to be filled, and the adapter may be connected to the needle shaft by an interference fit or threadedly.

Preferably, the adapter may be a quick-connect connector or a threaded connector.

The present invention provides the following benefits over the prior art: according to the present invention, through providing the pressure relief valve within the needle shaft at the second end thereof, accidental efflux of the solution being filled that may occur under a negative vacuum pressure, as well as splashing of the solution that may occur under a high negative vacuum pressure, can be avoided, and depressurization and pressure stabilization effects can be provided during filling of the solution. In particular, when filling of the liquid is stopped and an output pressure in the solution in the needle is eliminated, the elastic members in the pressure relief valve enable restoration of an internal structure of the pressure relief valve. This allows quantitative suction of the solution, avoiding it from stringing at the dispensing port of the liquid-filling needle. In this way, the filling process can be conducted in a more stable and more controllable manner, without the problems including environmental contamination due to efflux or dripping of the liquid being filled and variation in quality. Further, dripping caused by repeated vacuuming in repeated filling cycles of the liquid-filling needle can be addressed, and improved filling accuracy and quality can be achieved, making it very suitable for use in precise filling in environments with high vacuum levels.

### BRIEF DESCRIPTION OF THE DRAWINGS

FIG. 1 is a schematic structural overview of a vacuum liquid-filling needle according to an embodiment of the present invention;

FIG. 2 is a cutaway view of a pressure relief valve for use with a needle shaft according to an embodiment of the present invention;

FIG. 3 is a bottom view of the pressure relief valve for use with a needle shaft of FIG. 2;

FIG. 4 is a schematic structural overview of the pressure relief valve according to an embodiment of the present invention;

FIG. 5 is a schematic cutaway view of the pressure relief valve according to an embodiment of the present invention;

FIG. 6 is a schematic diagram showing the structure of a piston in the pressure relief valve according to an embodiment of the present invention;

FIG. 7 is a schematic diagram showing the structure of a diaphragm in the pressure relief valve according to an embodiment of the present invention;

FIG. 8 is a schematic diagram showing the structure of the vacuum liquid-filling needle with a flat dispensing head according to an embodiment of the present invention; and

FIG. 9 is a schematic diagram showing the structure of the vacuum liquid-filling needle with a flared dispensing head according to an embodiment of the present invention.

In these figures,

1 denotes an adapter; 2, a needle shaft; 3, a pressure relief valve; 4, a dispensing head; 21, a first end; 22, a second end; 30, a valve body; 31, a piston; 32, a diaphragm; 33, an inflow channel; 34, an inflow port; 35, an outflow port; 36, a damping aperture; 37, an outflow channel; 38, an exit aperture; 39, an annular groove; 300, an inter-cavity passage; 301, a first securing member; 302, a second securing member; 391, a first compression spring; 392, a second compression spring; 310, a piston body; 311, a first cavity; 312, a piston seat; 313, a first enclosing post; 314, a first protrusion; 315, a bevel; 320, a diaphragm body; 321, a second cavity; 322, a diaphragm seat; 323, a second enclosing post; 324, a second protrusion; 371, a suction aperture; 41, a flat dispensing head; 42, a flared dispensing head; and 411 and 421, dispensing ports.

### DETAILED DESCRIPTION

Objects, aspects and advantages of the present invention will become more apparent upon reading the following description of embodiments thereof set forth with reference to the accompanying drawings.

As used herein, the terms “internal”, “external”, “above”, “under” and the like are for illustrative purposes only and not intended as the only possible implementation. As used herein, the term “axial” refers to the direction of a center axis of a hydraulic valve. As used herein, the term “initial configuration” refers to a configuration where the filling of a solution has not started yet, with a pressure relief valve remaining inoperative.

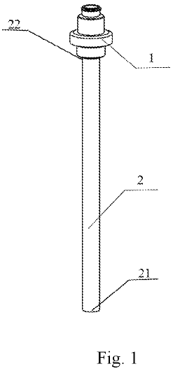

In an embodiment of the present invention, there is provided a vacuum liquid-filling needle, which includes an adapter 1, a needle shaft 2 and a pressure relief valve 3. As shown in FIG. 1, the needle shaft 2 includes a first end 21 and a second end 22. The adapter 1 is provided at the second end 22 and has a quick-connect or threaded port for connection with a pipe for supplying a solution to be filled. The adapter 1 is connected to the needle shaft 2 by an interference fit or threadedly. The pressure relief valve 3 is disposed within the needle shaft 2 at the first end 21. The pressure relief valve 3 defines an external thread by which it is threadedly coupled to the needle shaft 2. In alternative embodiments, the coupling between the pressure relief valve 3 and the needle shaft 2 may be accomplished by an interference fit, or otherwise.

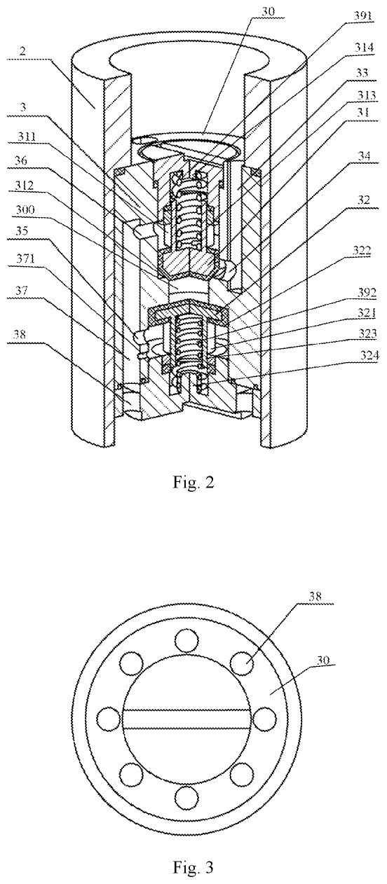

In this embodiment, as shown in FIGS. 2, 4 and 5, the pressure relief valve 3 includes a valve body 30 defining an internal cavity, in which a first slide valve and a second slide valve are disposed so as to be axially spaced apart from each other and both movable relative to the internal cavity. Each of the first and second slide valves may be a piston or diaphragm. This embodiment will be set forth in detail below in the context of the first slide valve being implemented as a piston 31 and the second slide valve as a diaphragm 32 as an example. In other embodiments, this may be flexibly configured, for example, with the first slide valve as a diaphragm and the second slide valve as a piston, or with both the first and second slide valves as pistons or diaphragms, without limiting the present invention in any sense.

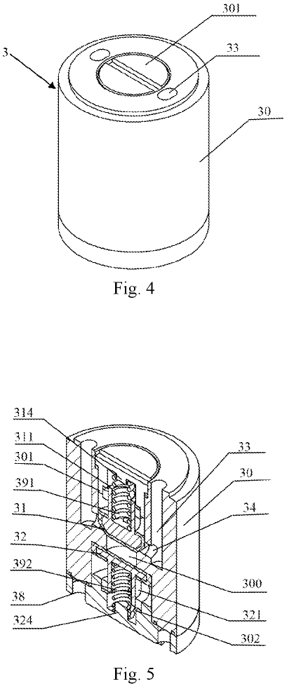

The valve body 30 defines an inflow channel 33, an inflow port 34, an outflow port 35, a damping aperture 36 and an outflow channel 37. The inflow channel 33 is provided by a blind bore for flowing therein of the solution to be filled, which extends axially along the valve body 30. Preferably, a plurality of inflow channels 33 are provided evenly around a circumference of the valve body 30. This embodiment is not limited to any particular number of inflow channels 33, or any particular number of inflow ports 34, and each of the numbers may be, for example, one, two, four, five, six, eight, or ten. In the embodiment shown in FIGS. 4 and 5, two inflow channels 33 are included. Each of the inflow channels 33 is open at a location closer to the second end 22 of the needle shaft 2. The inflow ports 34 are formed in a wall of the internal cavity so as to bring the respective inflow channels 33 into the internal cavity. The number of inflow ports 34 is the same as that of inflow channels 33.

Equally, the outflow channel 37 is provided by a blind bore for flowing out therefrom of the solution to be filled, which extends axially along the valve body 30. The pressure relief valve further includes an exit aperture 38 in communication with the outflow channel 37. The outflow channel 37 is open at a location closer to the first end 21 of the needle shaft 2. The outflow port 35 is formed also in the wall of the internal cavity so as to bring the outflow channel 37 into communication with the internal cavity. This embodiment is not limited to any particular number of outflow ports 35, or any particular number of outflow channels 37, or any particular number of exit apertures 38, and each of the numbers may be, for example, one, two, four, five, six, eight, or ten. Preferably, a plurality of outflow channels 37 are provided evenly around the circumference of the valve body 30. The number of outflow ports 35 and the number of exit apertures 38 may be the same as that of outflow channels 37. In this case, outlets of the outflow channels 37 may be brought into direct communication with the respective exit apertures 38. The number of exit apertures 38 may be different from, and preferably greater than, the number of outflow channels 37, in order to enable rapid efflux of the solution to be filled. In the embodiment shown in FIG. 3, two outflow channels 37 and eight exit apertures 38 are provided. Additionally, annular grooves 39 are provided between the outflow channels 37 and the exit apertures 38 to bring them into communication with each other. The plurality of exit apertures 38 are provided evenly around the circumference. The inflow ports 34 and the outflow ports 35 axially divide the internal cavity into, sequentially joined, a first cavity 311, an inter-cavity passage 300 and a second cavity 321.

The piston 31 is configured to be maintained in an initial configuration by a first elastic force at a first position where it resides over the inflow ports 34 to block communication between the inter-cavity passage 300 and the inflow channels 33. When subjected to a first axial pressure from the solution to be filled, which is greater than the first elastic force, the piston 31 will axially move against the action of the first elastic force from the first position toward the first cavity 311, bringing the inter-cavity passage 300 into communication with the inflow channels 33. When the first axial pressure from the solution to be filled drops below the first elastic force or disappears, the piston 31 will return to the first position under the action of the first elastic force, where it will again reside on the inflow ports 34 and block communication between the inter-cavity passage 300 and the inflow channels 33. In a preferred embodiment, the first position is where the inflow ports 34 come into communication with the inter-cavity passage 300. The diaphragm 32 is configured to be maintained in the initial configuration by a second elastic force at a second position where it blocks communication between the inter-cavity passage 300 between the piston 31 and the diaphragm 32 and the outflow channels 37. When subjected to a second axial pressure from the solution to be filled, which is greater than the second elastic force, the diaphragm 32 will axially move in the internal cavity against the action of the second elastic force from the second position toward the second cavity 321, bringing the inter-cavity passage 300 between the piston 31 and the diaphragm 32 into communication with the outflow channels 37. Likewise, when the second axial pressure from the solution to be filled drops below the second elastic force or disappears, the diaphragm 32 will return to the second position under the action of the second elastic force, where it will again block communication between the inter-cavity passage 300 between the piston 31 and the diaphragm 32 and the outflow channels 37. In a preferred embodiment, the second position lies between the first position and the outflow ports 35. The first axial pressure may be equal to the second axial pressure or not.

In this embodiment, as shown in FIGS. 2 and 5, the piston 31 is located above the diaphragm 32. Accordingly, the first cavity 311 is located above the inter-cavity passage 300, which is in turn located above the second cavity 321. The pressure relief valve 3 further includes a first securing member 301 and a second securing member 302. The internal cavity includes a third end and a fourth end. The first securing member 301 is fixed at the third end of the internal cavity, and the second securing member 302 at the fourth end of the internal cavity. The third end is closer to the second end 22 of the needle shaft 2 than the fourth end. Accordingly, the first cavity 311 is defined by both the first securing member 301 and the inflow ports 34, and the second cavity 321 is defined by both the second securing member 302 and the outflow ports 35.

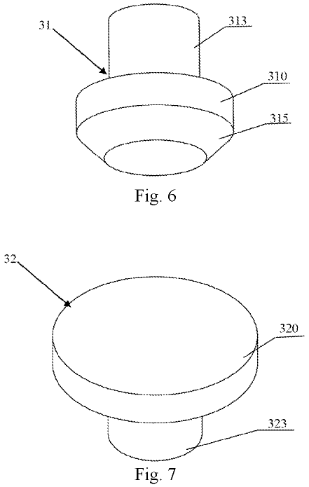

The pressure relief valve 3 further includes a first valve seat and a second valve seat. The first valve seat is configured to maintain the first slide valve at the first position and block the first slide valve from approaching the second slide valve. The second valve seat is configured to maintain the second slide valve at the second position and block the second slide valve from approaching the first slide valve. In this embodiment, the first position lies where the inflow ports 34 come into communication with the inter-cavity passage 300, and the second position is between the first position and the outflow ports 35. The first valve seat is a piston seat 312, and the second valve seat is a diaphragm seat 322. The piston seat 312 is configured to block the piston 31 from approaching the diaphragm 32, and the diaphragm seat 322 is configured to block the diaphragm 32 from approaching the piston 31. Specifically, the piston seat 312 is a first step defined in the valve body 30, and the first step has a shape matching with that of the piston 32. The diaphragm seat 322 is a second step defined in the valve body 30, and the second step has a shape matching with that of the diaphragm 32. Further, as shown in FIG. 6, the piston 31 has a bevel 315, which allows the first force exerted thereon by the solution to be filled to have an axial component.

With combined reference to FIGS. 2, 5 and 6, a first elastic member for providing the first elastic force is provided in the first cavity 311, which is, for example, a first compression spring 391. Specifically, the first compression spring 391 is brought into abutment with the piston 31 at one end and with the first securing member 301 at the other end. Additionally, the piston 31 includes a piston body 310 configured to abut, at the first position, against one end of the inflow channel 33 where it is joined to the inter-cavity passage 300, thereby blocking communication between the inter-cavity passage 300 and the inflow channels 33. In addition to the bevel 315, the piston body 310 further includes a hollow first enclosing post 313 extending toward the first securing member 301. The first enclosing post 313 has an inner diameter slightly greater than an outer diameter of the first compression spring 391 to allow the first compression spring 391 to be accommodated therein without radial movement, swinging or twisting. In addition, the first securing member 301 includes a first groove for receiving the first enclosing post 313. Further, a first protrusion 314 protrudes from a bottom of the first groove toward the piston 31, and an end portion of the first compression spring 391 is sleeved over the first protrusion 314. This additionally prevents radial movement, swinging and twisting of the first compression spring 391. In the initial configuration, a gap is left between an open end of the first enclosing post 313 and the bottom of the first groove to ensure axial movability of the piston 31.

With combined reference to FIGS. 2, 5 and 7, a second elastic member for providing the second elastic force is provided in the second cavity 321, which is, for example, a second compression spring 392. Specifically, the second compression spring 392 is brought into abutment with the diaphragm 32 at one end and with the second securing member 302 at the other end. Additionally, the diaphragm 32 includes a diaphragm body 320 defining a hollow second enclosing post 323 extending toward the second securing member 302. The second enclosing post 323 has an inner diameter slightly greater than an outer diameter of the second compression spring 392 to allow the second compression spring 392 to be accommodated therein without radial movement, swinging or twisting. In addition, the second securing member 302 includes a second groove for receiving the second enclosing post 323. Further, a second protrusion 324 protrudes from a bottom of the second groove toward the diaphragm 32, and an end portion of the second compression spring 392 is sleeved over the second protrusion 324. This additionally prevents radial movement, swinging and twisting of the second compression spring 392. In the initial configuration, a gap is left between an open end of the second enclosing post 323 and the bottom of the second groove to ensure axial movability of the diaphragm 32.

The damping aperture 36 is provided in a wall of the first cavity 311 so as to bring the outflow channels 37 into communication with the first cavity 311. The damping aperture 36 is provided to enable a stable pressure in the first cavity 311 between the piston 31 and the first securing member 301 while the piston 31 is moving. Preferably, a distance between the damping aperture 36 and the first position is greater than a distance between the open end of the first enclosing post 313 and the bottom of the first groove in order to prevent the piston 31 from blocking the damping aperture 36.

Additionally, a suction aperture 371 is provided in a wall of the second cavity 321 so as to bring the outflow channels 37 into communication with the second cavity 321. The suction aperture 371 is configured to allow discharge of the solution that is sucked back into the second cavity 321. After the diaphragm 32 returns to the second position, the solution to be filled will flow back into an internal cavity defined by the diaphragm 32 and the second securing member 302. If this cavity is not emptied, the diaphragm 32 will not be able to move toward the second securing member 302. In order to avoid this, the suction aperture 371 is provided in the wall of the second cavity 321. Preferably, the suction aperture 371 is provided at the end of the second securing member 302 proximal to the diaphragm 32.

During use of the pressure relief valve 3 according to this embodiment, the solution to be filled flows to the inflow ports 34 through the inflow channels 33 in the pressure relief valve 3 and exerts a first force on the piston 31. When the first force increases and overcomes the first elastic force, the piston 31 moves toward the first securing member 301 into the first cavity 311, bringing the inflow ports 34 into communication with the inter-cavity passage 300. As a result, the solution to be filled enters the inter-cavity passage 300. The solution to be filled in the inter-cavity passage 300 then exerts a second force on the diaphragm 32. When the second force increases and overcomes the second elastic force, the diaphragm 32 moves toward the second securing member 302 into the second cavity 321, bringing the outflow ports 35 into communication with the inter-cavity passage 300. As a result, the solution to be filled enters the outflow channel 37 and finally flows out from the exit apertures 38. Once the supply of the solution to the pressure relief valve 3 is cut off, the second force on the diaphragm 32 will drop below the second elastic force, and the diaphragm 32 will move toward the piston 31 and stay at the second position, blocking communication of the inter-cavity passage 300 between the piston 31 and the diaphragm 32 with the outflow ports 35. Upon the first force on the piston 31 dropping below the first elastic force, the piston 31 will move toward the diaphragm 32 and stay at the first position, blocking communication between the inflow ports 34 and the inter-cavity passage 300.

In this embodiment, the axial pressures from the solution are balanced with the elastic forces of the first and second elastic members, and a throttling effect is provided within the pressure relief valve 3. As a result, the solution to be filled is depressurized and prevented from being jetted from the needle shaft 2 when in a vacuum environment. In addition, when the piston 31 is moving toward the first securing member as a result of the outflow channels 37 being brought into communication with the damping aperture 36, the first cavity 311 is depressurized through the outflow channels 37. Additionally, a pressure at the exit apertures 38 is fed back, through the outflow channels 37, to the first cavity 311 and hence to the piston 31. Therefore, the use of the damping aperture 36 enables a stable output pressure of the solution to be filled during axial reciprocation of the piston 31. Once the filling is stopped, the liquid in the supply pipe will be totally depressurized, and the piston 31 and the diaphragm 32 will be restored to the first and second positions by the first and second elastic forces, respectively. At the same time as the restoration, the solution will be sucked back through the outflow ports 35, avoiding it from stringing at a dispensing tip of the liquid-filling needle. In this way, dripping caused by repeated vacuuming in repeated filling cycles of the liquid-filling needle can be greatly reduced, and improved filling accuracy and quality can be achieved.

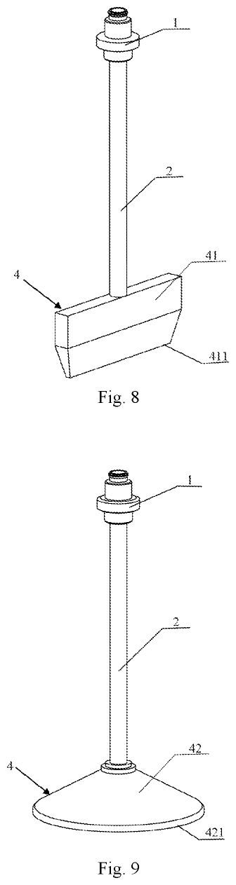

More preferably, a dispensing head in any of various shapes such as flat, cuboid, torus-like, flared and the like may be provided at the first end 21 of the needle shaft 2. The dispensing head 4 may have a dispensing port with a cross-sectional shape which may be square, rectangular, circular, etc. In case of a square cross-section, it is preferred to have a side length in the range of 10-200 mm. In case of a rectangular cross-section, it is preferred to have a length in the range of 10-400 mm and a width in the range of 0.05-200 mm. In case of a circular cross-section, it is preferred to have a diameter in the range of 10-200 mm. FIG. 8 shows an example of a flat dispensing head 41 that has a dispensing port 411 having an elongate rectangular cross-sectional shape, which allows a large dispensing width. The cross-sectional shape of the dispensing port 411 is preferred to have a length of 10-400 mm and a width of 0.05-5 mm. FIG. 9 shows an example of a flared dispensing head 42 that has a dispensing port 421 having a circular cross-sectional shape with a relatively large diameter, which allows the solution to be uniformly dispensed in a circular area with a predetermined diameter. The various shapes of the dispensing head allow the solution to be dispensed in different forms that meet the requirements of various applications.

Thus, the vacuum liquid-filling needle provided in the present invention, which incorporates the special pressure relief valve, can prevent undesired accidental efflux of the liquid under a negative vacuum pressure while providing depressurization and pressure stabilization effects during filling of the liquid. When filling of the liquid is stopped and an output pressure in the liquid in the liquid-filling needle is eliminated, an internal structure of the pressure relief valve can be restored and quantitative suction of the liquid can be achieved, avoiding it from stringing at the dispensing port of the liquid-filling needle. In this way, dripping caused by repeated vacuuming in repeated filling cycles of the liquid-filling needle can be addressed, and improved filling accuracy and quality can be achieved.

While the present invention has been described above in terms of preferred embodiments, it is not limited to the embodiments disclosed herein. Any person of skill in the art can make changes and modifications without departing from the scope or spirit of the invention. Accordingly, the true scope of the invention is intended to be as defined by the appended claims.

1. A pressure relief valve, comprising a valve body, the valve body defining an internal cavity, in which a first slide valve and a second slide valve are disposed so as to be axially spaced apart from each other and both movable relative to the internal cavity, the valve body defining an inflow channel, an inflow port, an outflow port and an outflow channel, each of the inflow channel and the outflow channel formed by a blind bore, both the inflow channel and the outflow channel axially extending along the valve body,
the inflow channel brought into communication with the internal cavity through the inflow port, the outflow channel brought into communication with the internal cavity through the outflow port, the inflow port and the outflow port dividing the internal cavity axially into a first cavity, an inter-cavity passage and a second cavity,
the first slide valve configured to be maintained in an initial configuration by a first elastic force at a first position where the first slide valve resides on the inflow port to block communication of the inter-cavity passage with the inflow channel and to, when subjected to a first axial pressure from a solution to be filled, which is greater than the first elastic force, move axially against the first elastic force from the first position toward the first cavity to bring the inter-cavity passage into communication with the inflow channel,
the second slide valve configured to be maintained in the initial configuration by a second elastic force at a second position where the second slide valve blocks communication of the inter-cavity passage between the first and second slide valves with the outflow channel and to, when subjected to a second axial pressure from the solution to be filled, which is greater than the second elastic force, move axially within the internal cavity against the second elastic force from the second position toward the second cavity to bring the inter-cavity passage between the first and second slide valves into communication with the outflow channel

2. The pressure relief valve of claim 1, wherein the first slide valve is further configured to, when the first axial pressure from the solution to be filled is below the first elastic force or disappears, return to the first position under an action of the first elastic force, and
the second slide valve is further configured to, when the second axial pressure from the solution to be filled is below the second elastic force or disappears, return to the second position under an action of the second elastic force.

3. The pressure relief valve of claim 1, wherein a damping aperture is provided in a wall of the first cavity so as to bring the outflow channel into communication with the first cavity.

4. The pressure relief valve of claim 1, further comprising a first securing member and a second securing member, the internal cavity having a third end and a fourth end, the first securing member fixed at the third end of the internal cavity, the second securing member fixed at the fourth end of the internal cavity, wherein:
a first elastic member for providing the first elastic force is provided in the first cavity so as to abut against the first slide valve at an end and against the first securing member at a further end; and
a second elastic member for providing the second elastic force is provided in the second cavity so as to abut against the second slide valve at an end and against the second securing member at a further end.

5. The pressure relief valve of claim 4, wherein the first slide valve comprises a first slide valve body comprising a hollow first enclosing post extending axially within the first cavity, the first elastic member accommodated in the first enclosing post, and wherein the second slide valve comprises a second slide valve body comprising a hollow second enclosing post extending axially within the second cavity, the second elastic member accommodated in the second enclosing post.

6. The pressure relief valve of claim 5, the first securing member defines a first groove for receiving the first enclosing post, and the second securing member defines a second groove for receiving the second enclosing post.

7. The pressure relief valve of claim 6, wherein a first protrusion extends from the first groove toward the first slide valve, and the further end of the first elastic member is sleeved over the first protrusion, and wherein a second protrusion extends from the second groove toward the second slide valve, and the further end of the second elastic member is sleeved over the second protrusion.

8. The pressure relief valve of claim 7, wherein a damping aperture is provided in a wall of the first cavity so as to bring the outflow channel into communication with the first cavity, and
wherein the damping aperture is spaced from the first position by a distance that is greater than a distance from an open end of the first enclosing post to a bottom of the first groove.

9. The pressure relief valve of claim 1, wherein the valve body is provided thereon with a first valve seat and a second valve seat, the first valve seat configured to maintain the first slide valve at the first position and block the first slide valve from approaching the second slide valve, the second valve seat configured to maintain the second slide valve at the second position and block the second slide valve from approaching the first slide valve.

10. The pressure relief valve of claim 1, wherein the valve body is further provided therein with a suction aperture, through which the outflow channel comes into communication with the internal cavity, and wherein the suction aperture is located between the outflow port and an outlet of the outflow channel

11. The pressure relief valve of claim 1, wherein the first slide valve is a piston or a diaphragm, and the second slide valve is a piston or a diaphragm.

12. The pressure relief valve of claim 11, wherein the first slide valve is a piston with a bevel for enabling the solution to be filled to exert the first axial pressure on the piston.

13. The pressure relief valve of claim 1, wherein the pressure relief valve further comprises exit apertures and an annular groove, a number of the exit apertures being greater than a number of the outflow channels, the exit apertures communicating with the outflow channels through the annular groove.

14. A vacuum liquid-filling needle, comprising a needle shaft and a pressure relief valve, the pressure relief valve disposed within the needle shaft at a first end thereof, the needle shaft being able to connect at a second end thereof to a pipe for supplying a solution to be filled, the pressure relief valve is defined in claim 1, wherein the inflow channel is open at a location closer to the second end of the needle shaft, and the outflow channel is open at a location closer to the first end of the needle shaft.

15. The vacuum liquid-filling needle of claim 14, wherein the pressure relief valve is connected to the needle shaft by an interference fit or threadedly.

16. The vacuum liquid-filling needle of claim 14, wherein a dispensing head is provided at the first end of the needle shaft.

17. The vacuum liquid-filling needle of claim 16, wherein the dispensing head comprises a dispensing port having a cross-section in shape of a square, a rectangle or circle, the square having a side length in a range of 10 mm to 200 mm, the rectangle having a length in a range of 10 mm to 400 mm and a width in a range of 0.05 mm to 200 mm, the circle having a diameter in a range of 10 mm to 200 mm

18. The vacuum liquid-filling needle of claim 16, wherein the dispensing head is detachably connected to or integral with the needle shaft.

19. The vacuum liquid-filling needle of claim 14, wherein the needle shaft is provided at the second end thereof with an adapter for connecting to the pipe for supplying the solution to be filled, the adapter connected to the needle shaft by an interference fit or threadedly.

20. The vacuum liquid-filling needle of claim 19, wherein the adapter is a quick-connect connector or a threaded connector.
